(19) United States
(12) Patent Application Publication
(10) Pub. No.: US 2010/0160808 A1
(43) Pub. Date: Jun. 24, 2010

(54) INTERFACE SYSTEM UTILIZING MUSTICATORY ELECTROMYOGRAM

(76) Inventors: Shinobu ADACHI, Osaka (JP); Koji Morikawa, Kyoto (JP)

Correspondence Address: MARK D. SARALINO (PAN);RENNER, OTTO, BOISSELLE & SKLAR, LLP, 1621 EUCLID AVENUE, 19TH FLOOR, CLEVELAND, OH, 44115, US

(21) Appl. No.: 12/715,447

(22) Filed: Mar. 2, 2010

Related U.S. Application Data
(63) Continuation of application No. PCT/JP2009/003078, filed on Jul. 2, 2009.

(30) Foreign Application Priority Data
Jul. 11, 2008 (JP) 2008-181670

(51) Int. Cl. A61B 5/0488; G06F 3/048

(52) U.S. Cl. 600/546; 715/821; 715/811; 715/825

(57) ABSTRACT

The interface system includes: an output section for visually presenting a manipulation menu for a device; a measurement section for measuring a masticatory electromyogram of a user; a menu presentation section for causing menu items of the manipulation menu to be presented in order via the output section; an amplitude calculation section for determining a maximum amplitude of a potential waveform of the masticatory electromyogram; a latency calculation section for determining a latency based on a point of highlighting each menu item as a starting point, the latency being an amount of time in which the potential waveform arrives at the maximum amplitude; and a determination section for determining whether the maximum amplitude is greater than a previously determined threshold value and the latency is approximately 200 ms or not. In accordance with the determination result of the determination section, the menu presentation section executes a process corresponding to the highlighted menu item.

This is a continuation of International Application No. PCT/JP2009/003078, with an international filing date of Jul. 2, 2009, which claims priority of Japanese Patent Application No. 2008-181670, filed on Jul. 11, 2008, the contents of which are hereby incorporated by reference.

### BACKGROUND OF THE INVENTION

1. Field of the Invention

The present invention relates to an interface system for a device. More specifically, the present invention relates to an interface system for a device where the interface system utilizes biological information (an electromyogram which can be measured in the neighborhood of the head) of a user.

2. Description of the Related Art

In recent years, various types of information devices such as television sets, mobile phones, and head-mount displays (HMDs) have gained prevalence and entered into people's lives. Therefore, users need to manipulate information devices in many scenes of their usual lives.

Usually, a user uses a hand to input an input command via an input means (interface section) such as a button, thus realizing a manipulation of a device. However, in situations where both hands are full because of tasks other than a device manipulation, e.g., household chores, rearing of children, or driving, it is difficult to manipulate the interface section by using a hand and it is impossible to realize a device manipulation. Moreover, a user who is not able to freely move his or her hands due to damaging of the cervical spinal cord, etc., would have difficulties in manipulating a button by using a hand. Therefore, there are increasing needs of users to manipulate a device in a handsfree manner.

Against such needs, there have been developed input means that utilize an electromyogram which occurs due to an activity of masticatory muscles (related to motions of the mandible) and which is measured in the neighborhood of the head as a biological signal (hereinafter referred to as an “masticatory electromyogram” in the present specification).

A masticatory electromyogram also occurs responsive to any motion of the temporomandibular joints caused during conversations or by mastication associated with eating in daily life. Therefore, in order to realize an interface utilizing a masticatory electromyogram, it has been necessary to discern masticatory electromyograms that occur in daily life from a masticatory electromyogram that appears due to an activity of the masticatory muscles which a user has intentionally made for a manipulation. However, whether intentional or not, there is no difference in the characteristic features (frequency, amplitude, etc.) of a masticatory electromyogram that appears due to a chewing motion, and therefore it has been difficult to exclusively detect a masticatory electromyogram which has been intentionally issued, within a masticatory electromyogram that is continuously measured over a predetermined period.

Therefore, for example, Japanese Laid-Open Patent Publication No. 2007-125380 (hereinafter to as “Patent Document 1”) discloses a technique where a user is supposed to perform a special chewing motion which would never occur in daily life, so that only an intentional masticatory electromyogram will be detected for use as a control signal for a wheelchair. This technique is based on the premise that a special masticatory electromyogram which occurs responsive to a special chewing motion is discernable from a masticatory electromyogram of daily life.

In Patent Document 1, the user is supposed to perform a special chewing motion such as: (1) a sustained chewing motion for several hundred milliseconds (hereinafter “ms”) or more; and (2) a chewing motion on only the left side or only the right side. Then, an intentional masticatory electromyogram which has occurred responsive to such a special chewing is detected by a method of applying threshold processing to a difference value between potentials which are measured at a plurality of electrodes attached to the right and left sides of the forehead. By utilizing this special chewing method, in controlling a powered wheelchair, (1) a sustained manipulation signal can be generated, so that a sustained travel can be realized (that is, it can specify a time zone during which the signal is ON, and can be employed as an accelerator), and (2) a right or left chew corresponds to a right or left pivoting of the powered wheelchair, whereby a comprehensible input method is provided. Thus, an interface that presents little burden on the user is realized.

On the other hand, some input means have been developed that distinguish an option which the user wants to select by utilizing an event-related potential of an electroencephalogram as a biological signal. Emanuel Donchin and two others, “The Mental Prosthesis: Assessing the Speed of a P300-Based Brain-Computer Interface”, IEEE TRANSACTIONS ON REHABILITATION ENGINEERING, Vol. 8, June 2000 (hereinafter referred to as “Non-Patent Document 1”) discloses a technique of detecting an option which a user wants to select by utilizing a P3 component of an event-related potential that appears about 300 ms after a timing of highlighting the option as a starting point, among randomly highlighted options. This technique makes it possible for the user to select an option which he or she wants to select, without using a hand.

However, the input method utilizing a masticatory electromyogram which is described in Patent Document 1 requires the user to perform a special (sustained and characteristic) chewing motion so that an intentional masticatory electromyogram can be discerned from masticatory electromyograms of daily life. As a result, there have been problems in that: (1) a manipulation input cannot be made in a short time; and (2) there is a limit to the number of commands because of the limited kinds of special chewing. These problems are worsened especially when the method is applied to an interface of an information device which requires real-time manipulation inputs and a large number of commands, e.g., television sets, mobile phones, HMDs, or the like.

On the other hand, the input method utilizing an electroencephalogram which is described in Non-Patent Document 1 has an advantage in that the user is able to select all commands with the same method because a menu item can be selected merely by outputting a user's intention of selection (1-bit information) as to whether a highlighted menu item is meant to be selected or not.

However, in order to distinguish an event-related potential having a low S/N, it is necessary to take an arithmetic mean over 5 to 10 times (repetitive highlighting). Thus, there is a problem in that a reliable menu selection cannot be made in a short time.

Furthermore, there is the problem of positions to wear electrodes. In Patent Document 1, in order to detect a difference between the right and left chews, a plurality of electrodes must be worn on the right and left sides of the forehead; and in Non-Patent Document 1, at least one electrode must be worn at the parietal for detecting a P3 component. Wearing of such electrodes has presented a burden to the user.

### SUMMARY OF THE INVENTION

An objective of the present invention is to provide an interface system which enables a rapid menu selection even in a situation where masticatory electromyograms of daily life may occur, e.g., during conversation or while eating, without having to repeat any special chewing motion or highlighting.

An interface system according to the present invention comprises: an output section for visually presenting a manipulation menu for a device; a measurement section for measuring a masticatory electromyogram of a user; a menu presentation section for causing menu items of the manipulation menu to be presented in order via the output section; an amplitude calculation section for determining a maximum amplitude of a potential waveform of the masticatory electromyogram; a latency calculation section for determining a latency based on a point of highlighting each menu item as a starting point, the latency being an amount of time in which the potential waveform arrives at the maximum amplitude; and a determination section for determining whether the maximum amplitude is greater than a previously determined threshold value and the latency is approximately 200 ms or not, wherein, in accordance with a result of determination by the determination section, the menu presentation section executes a process corresponding the highlighted menu item.

If the determination section determines that the maximum amplitude is greater than the previously determined threshold value and the latency is in the neighborhood of 200 ms, it may be determined that the masticatory electromyogram has been intentionally issued, and the menu presentation section may execute the process corresponding to the highlighted menu item.

The measurement section may measure the masticatory electromyogram based on a potential difference measured with electrodes worn on a nasion and a mastoid of the user.

The interface system may further comprise a cut-out section for cutting out a potential waveform of the masticatory electromyogram in accordance with highlighting of each menu item by the menu presentation section, wherein the amplitude calculation section may determine the maximum amplitude of the potential waveform having been cut out.

The cut-out section may at least cut out a time slot including 150 ms to 250 ms after highlighting, when a masticatory electromyogram with respect to the highlighting is to appear.

If the maximum amplitude is greater than the previously determined threshold value and the latency is in the neighborhood of 200 ms, the determination section may determine that the measured masticatory electromyogram has been intentionally issued with respect to the highlighting of the menu item, and if the maximum amplitude is greater than the previously determined threshold value but the latency is not in the neighborhood of 200 ms, the determination section may determine that the measured masticatory electromyogram has not been intentionally issued with respect to the highlighting of the menu item.

Based on whether the maximum amplitude is greater than 50 μV as the previously determined threshold value and the latency is in the neighborhood of 200 ms or not, the determination section may determine that the measured masticatory electromyogram has been intentionally issued with respect to the highlighting of the menu item.

The determination section may acquire an average value and a variance value of each of maximum amplitude and latency of the user, and, based on the acquired average values and variance values, determine that the measured masticatory electromyogram has been intentionally issued with respect to the highlighting of the menu item, by using as a detection target range a range which is defined by the average value±a standard deviation of each.

The determination section may determine that the measured masticatory electromyogram has been intentionally issued with respect to the highlighting of the menu item, by using as detection target ranges which are defined by the acquired maximum amplitude ±50 μV and the acquired latency ±50 ms.

An order of menu item highlighting may be switched between descending and random in the menu presentation section, and the interface system may comprise a switching section for switching detection target ranges in the determination section based on which one of the descending order and the random order the menu items have been switched to in the menu presentation section.

If the order of menu item highlighting is random, the switching section may switch the detection target ranges for maximum amplitude and latency to be 200±70 μV and 280±50 ms, respectively.

The interface system may comprise a range determination section for retaining an elapsed time since a menu was last presented by the menu presentation section, and if a certain amount of time or more has elapsed, updating detection target ranges in the determination section in a direction of expanding the detection target ranges.

If the masticatory electromyogram is detected after the detection target ranges are expanded by the range determination section, the range determination section may newly set a maximum amplitude and a latency value of the detected masticatory electromyogram as central values of the detection target ranges.

A method according to the present invention is a method to be executed in an interface system, the method comprising the steps of: visually presenting a manipulation menu for a device; receiving a measured masticatory electromyogram of a user; presenting menu items of the manipulation menu in order via the output section; determining a maximum amplitude of a potential waveform of the masticatory electromyogram; determining a latency based on a point of highlighting each menu item as a starting point, the latency being an amount of time in which the potential waveform arrives at the maximum amplitude; determining whether the maximum amplitude is greater than a previously determined threshold value and the latency is approximately 200 ms or not; and executing a process corresponding to the highlighted menu item in accordance with a result of determination by the determining step.

A computer program to be executed by a computer according to the present invention causes the computer to execute the steps of: visually presenting a manipulation menu for a device; receiving a measured masticatory electromyogram of a user; presenting menu items of the manipulation menu in order via the output section; determining a maximum amplitude of a potential waveform of the masticatory electromyogram; determining a latency based on a point of highlighting each menu item as a starting point, the latency being an amount of time in which the potential waveform arrives at the maximum amplitude; determining whether the maximum amplitude is greater than a previously determined threshold value and the latency is approximately 200 ms or not; and executing a process corresponding to the highlighted menu item in accordance with a result of determination by the determining step.

In accordance with an interface system of the present invention, a potential change (masticatory electromyogram) in the neighborhood of the head, which is cut out based on the highlighting of a menu item as a starting point, is measured. Based on whether the maximum amplitude of the masticatory electromyogram is greater than a previously determined threshold value (e.g. 50 μV) and the latency of the masticatory electromyogram is in the neighborhood of 200 ms or not, it is determined as to whether the measured masticatory electromyogram has been intentionally issued with respect to the highlighting of the menu item or not. As a result, there is realized a handsfree interface which enables a rapid menu selection even in a situation where masticatory electromyograms of daily life may occur, e.g., during conversation or while eating, without having to repeat any special chewing motion or highlighting.

Other features, elements, processes, steps, characteristics and advantages of the present invention will become more apparent from the following detailed description of preferred embodiments of the present invention with reference to the attached drawings.

### BRIEF DESCRIPTION OF THE DRAWINGS

FIG. 1 is a diagram showing in chronological order a method of highlight-type menu presentation.

FIG. 2 is a diagram showing masticatory muscles 201 which exist in the neighborhood of the head of a human body.

FIGS. 3A to 3D are diagrams showing positions of electrodes to be worn for measuring a masticatory electromyogram.

FIG. 4 is a flowchart showing a processing procedure of a device which performs highlight-type menu presentation and potential measurement during an experiment conducted by the inventors.

FIG. 5A is a simplified diagram showing menu items which were actually presented to test subjects.

FIG. 5B is a diagram showing an example of highlighting.

FIG. 6A is a diagram showing a flow of a chewing-selection condition on the participant side.

FIG. 6B is a flowchart showing a processing procedure under speaking/eating conditions on the participant side.

FIGS. 7A to 7C are diagrams showing exemplary experimental results.

FIG. 8A is a diagram showing potential waveforms under the chewing-selection condition, which are cut out for respective highlights that are accompanied or not accompanied by an intentional chewing motion, plotted by using latency and maximum amplitude as characteristic amounts.

FIG. 8B is a diagram showing masticatory electromyograms when an intentional chewing is made under the chewing-selection condition and masticatory electromyograms under the speaking and eating conditions, plotted by using latency and maximum amplitude as characteristic amounts.

FIG. 9 is a diagram showing a construction and environment of use for an interface system 1 which utilizes a masticatory electromyogram.

FIG. 10 is a diagram illustrating an example where a TV 2 is manipulated in the interface system 1 by a user 5 who selects and watches a program which he or she wishes to view.

FIG. 11 is a diagram showing a functional block construction of the interface system 1 according to Embodiment 1.

FIG. 12 is a flowchart showing a processing procedure of the interface system 1, which performs determination as to whether a user made a short-time intentional chewing motion immediately after menu item highlighting.

FIG. 13 is a flowchart showing a detailed processing procedure of a masticatory electromyogram detector which determines whether or not the user 5 made an intentional chewing motion immediately after menu item highlighting.

FIG. 14 is a diagram showing results of a chewing-selection condition in a random experiment and a descending-order experiment, plotted by using latency and maximum amplitude as characteristic amounts.

FIG. 15 is a diagram showing a functional block construction of an interface system 2 according to Embodiment 2.

FIG. 16 is a flowchart showing the processing procedure of the interface system 2 according to Embodiment 2.

FIG. 17 is a diagram showing results of performing a descending-order experiment for the same experimental participants on different days, plotted by using latency and maximum amplitude as characteristic amounts.

FIG. 18 is a diagram showing a functional block construction of an interface system 3 according to Embodiment 3.

FIG. 19 is a flowchart showing the processing procedure of the interface system 3 according to Embodiment 3.

### DETAILED DESCRIPTION OF PREFERRED EMBODIMENTS

Hereinafter, with reference to the accompanying drawings, embodiments of an interface system utilizing a masticatory electromyogram according to the present invention will be described.

The inventors have found, in a situation where menu items are highlighted one by one, the menu items being presented in connection with device manipulations to be executed, a method of precisely detecting an intentional chewing motion which a user makes simultaneously with or immediately after the highlighting of a menu item meant to be selected. Specifically, the inventors have found that, if a masticatory electromyogram is measured from the potentials of the nasion and the mastoids of the user (i.e., positions at which a pair of glasses will be touching the ears when the glasses are worn) when the user makes an intentional chewing motion simultaneously with or immediately after highlighting of a menu item meant to be selected, a maximum amplitude and a latency of the potential waveform of a masticatory electromyogram which is cut out based on the highlighting of the menu item as a starting point will stably appear in the neighborhood of 100 μV and in the neighborhood of 200 ms, respectively. It has also been found that, by relying on the maximum amplitude and latency as indices, a distinction from masticatory electromyograms of daily life can be made. As a result, based on a masticatory electromyogram, it is possible to distinguish an intentional chewing motion of a user and detect an intention of selection of the user.

One expression which the present specification will employ is “the neighborhood of” a value X, e.g., “in the neighborhood of 100 μV” and “in the neighborhood of 200 ms”; this may also be expressed as “approximately” X. In either case, it is meant that a range containing the value X is encompassed. The expanse of the range needs to be determined based on complex factors such as individual differences in each biological signal corresponding to the value, measurement errors of the value of interest, and the like. In order to detect whether or not such a biological signal has appeared, a “detection target range” is defined in the embodiments. This detection target range may be considered as “the neighborhood of X”. For example, if the maximum amplitude of the potential waveform of a masticatory electromyogram is “in the neighborhood of 100 μV”, the “neighborhood” may mean 100±50 μV. If the latency is “in the neighborhood of 200 ms”, the “neighborhood” may mean a time slot which includes 200±50 ms.

Hereinafter, a method of highlight-type menu presentation will be described first, followed by a description of experiments which the inventors have performed in order to investigate in characteristic features of a masticatory electromyogram that appears responsive to an intentional chewing motion with respect to the highlighting of a menu item meant to be selected, as well as the inventors' findings concerning characteristic features of an intentional masticatory electromyogram for a menu item highlighting.

Thereafter, as Embodiments, an outline of an interface system which performs highlight-type menu presentation, as well as the construction and operation of an interface system which includes a masticatory electromyogram detector, will be described.

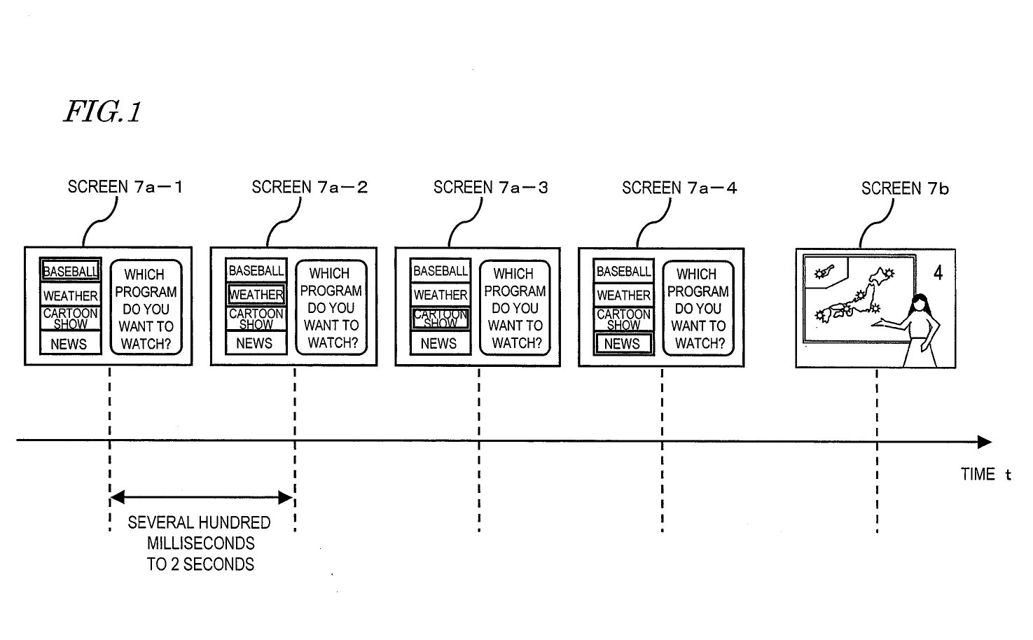

FIG. 1 is a diagram showing in chronological order a method of highlight-type menu presentation. Screens 7a-1 to 7a-4 illustrate four items of a device's manipulation menu being highlighted one by one.

In the present specification, the group of options concerning device manipulations as shown in FIG. 1 is referred to as a “menu”; each option is referred to as a “menu item”; and a menu item which the user wants to select is referred to as an “item meant to be selected”.

As menu items are highlighted on the screen in this manner, the user can select a menu item by simply making an output concerning presence or absence (1-bit information) of the user's intention of selection with respect to a highlighted menu item.

Now, in order to rapidly make a menu selection during highlight-type menu presentation, two conditions are necessary: (1) highlighting is not repeated; and (2) a short highlighting interval is set.

For example, it is possible to detect a user's intention of selection by utilizing a P3 component of an event-related potential as described in Non-Patent Document 1. Herein, an “event-related potential” refers to a transient potential fluctuation in the brain which occurs in temporal relationship with an external or internal event. Generally speaking, a “P3 component” component of an event-related potential is often regarded as a positive component of the event-related potential which appears near approximately 300 ms from the starting point, regardless of the type of sensory stimulation such as auditory sense, visual sense, or somatic sensation.

In the case where a P3 component is utilized, it is known that an intention of selection of an experienced user is detectable even with a highlighting time interval of 125 ms. In the case where the number of menu items is four, the time which will be required for all menu items to be highlighted once is as short as 500 ms. However, for an accurate detection of an intention of selection, repetitive highlighting will be needed because a P3 component of an event-related potential has a low S/N. For example, if highlighting is repeated five times, as much time as 2.5 seconds will be required for outputting an intention of selection.

On the other hand, there have been no instances of measuring a masticatory electromyogram with respect to highlight-type menu presentation. However, as in Patent Document 1, a method of detecting a masticatory electromyogram which occurs responsive to a special (sustained and characteristic) chewing motion and selecting a highlighted menu item is also conceivable. This will provide an advantage in that repetitive highlighting for taking an arithmetic mean is unnecessary because a masticatory electromyogram has a high S/N.

However, because the temporal relationship between the onset of a highlight and a special masticatory electromyogram caused by an intentional mastication is not known, it is presumable that a short highlighting interval cannot be chosen. Therefore, 1 second or more will be required, for example. Therefore, it is considered that rapid menu selection is difficult. Furthermore, since a prolonged chewing motion which would not occur in daily life is the target of detection, there is a foreseeable problem of delayed detection of the intent of manipulation.

Therefore, in order to clarify characteristic features of a masticatory electromyogram which appears when an intentional chewing motion is made immediately after a highlighting during highlight-type menu presentation, and in particular to clarify its temporal relationship with the onset of a highlight, the inventors have performed experiments to investigate in the characteristic features of a masticatory electromyogram, under a condition of making a short-time chewing motion immediately after highlighting of an item meant to be selected. As a result, it has been found that, when an intentional mastication is made upon highlighting of an item meant to be selected, a masticatory electromyogram stably appears with a maximum amplitude in the neighborhood of 100 μV and in the neighborhood of 200 ms after highlighting. It has also been found that, by using as characteristic amounts the maximum amplitude and latency of a potential waveform which is cut out based on the highlighting as a starting point, a masticatory electromyogram (non-cumulative) which is intentionally issued upon highlighting of an item meant to be selected becomes discernable from masticatory electromyograms occurring in daily life. Thus, there is realized an interface system which enables a rapid menu selection even in a situation where masticatory electromyograms of daily life may occur, e.g., during conversation or while eating, without having to repeat any special chewing motion or highlighting.

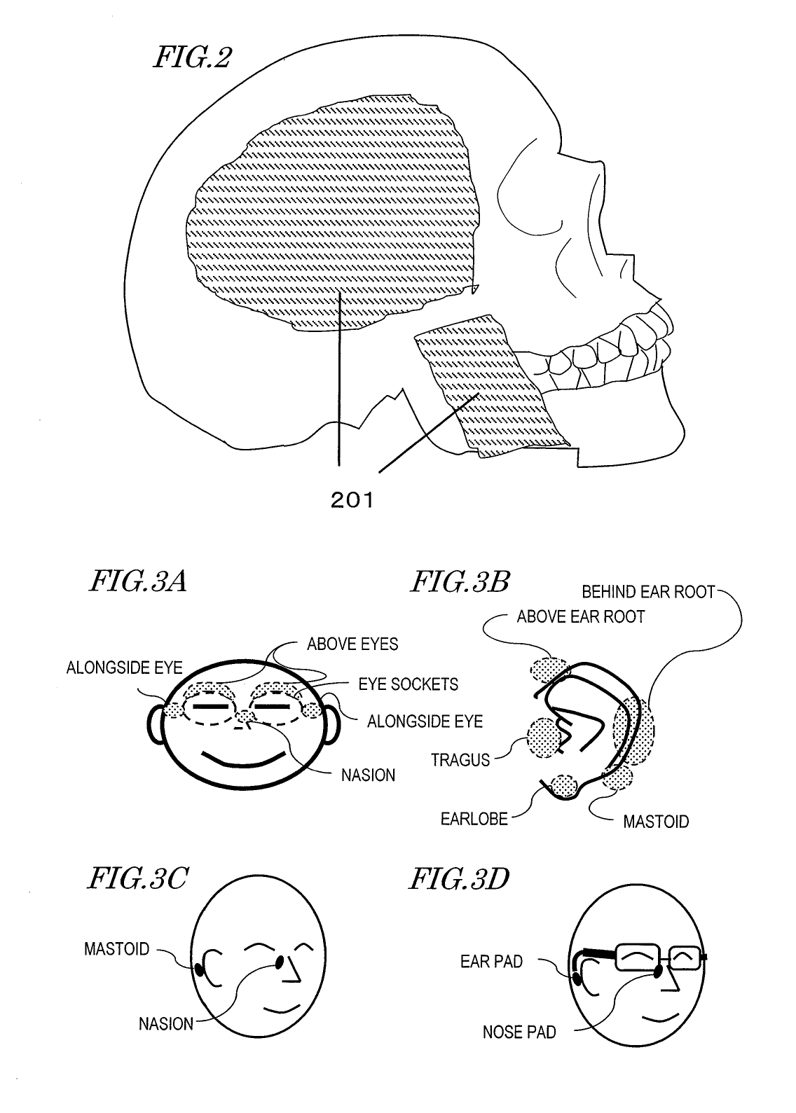

Hereinafter, with reference to FIG. 2 to FIG. 6, an experiment and experimental results will be described. The experiment was performed for two experimental participants (male).

FIG. 2 shows masticatory muscles 201 existing in the neighborhood of the head of a human body. The masticatory muscles 201 are, collectively, the masseter, the temporalis, the external pterygoid, and the internal pterygoid, although not so distinguished in FIG. 2.

Next, positions where electrodes are to be worn for measuring a masticatory electromyogram with respect to the masticatory muscles 201 described above will be described. The electrodes may be worn at various positions in the neighborhood of the head of a human body.

For example, as shown in FIGS. 3A and 3B, the electrodes may be worn above the eyes (upper edges of the eye sockets), alongside the eyes (outer edges of the eye sockets (outer corners of the eye lids)), the nasion, and above the ears (above the ear roots).

It is assumed that a masticatory electromyogram measuring function is conferred to a so-called head-mount display (HMD). When taking into consideration the HMD shape and the space which it occupies, in addition to the facial electrodes which are currently used for electro-oculographic potential measurement, locations in the ear periphery are also usable as targets of measurement in the neighborhood of the head, e.g., the earlobes, opisthotic (behind the ear roots), infraotic (under the ear roots), and prootic. FIG. 3B shows an earlobe, a mastoid, a tragus, the rear portion of the ear root, and the like.

As a representative of the aforementioned locations in the ear periphery, the inventors have chosen a mastoid, which is a protrusion of the cranium at the hind root of an ear, and conducted an experiment of evaluating the distinction ratio of an interface system for the conventionally-employed electrode positions which are on the face, relative to a reference electrode worn at the mastoid.

FIG. 3C is a diagram showing positions where electrodes are to be worn for potential measurement. The electrodes were worn at the nasion and a mastoid, and a potential of the nasion relative to the mastoid was measured. The nasion and the mastoids are portions which come in contact with a nose pad and ear pads when glasses are worn, and thus are positions where measurements can be naturally taken, with a glasses-type device being worn (FIG. 3D). Although not shown in FIG. 3C, a system reference electrode was worn at the mastoid opposite from the reference electrode.

The positions of wearing the electrodes are chosen astride the masticatory muscles. “Astride” means that one electrode is located on the skin over the masticatory muscles while the other electrode is located on the skin at a position where no masticatory muscles exist. When the electrodes are worn under such conditions, a masticatory electromyogram caused by an activity of the masticatory muscles can be surely measured. Note, however, that the aforementioned positions of wearing the electrodes are exemplary; other electrode positions astride the masticatory muscles may also exist that enable detection of a masticatory electromyogram. Potential changes were measured with a sampling frequency of 200 Hz and a time constant of 1 second.

A highlight-type menu was presented in which four menu items shown in FIG. 1 were highlighted in descending order as visual stimulations. The highlighting interval was 350 ms. The visual stimulations were presented on a 37″ plasma display, which was disposed 2 m before the test subject.

In order to examine whether or not a masticatory electromyogram occurring due to an intentional chewing motion made upon highlighting of a menu item can be discerned from masticatory electromyograms occurring in daily life, the experiment was performed under varying conditions. In each experiment, a similar highlight-type menu presentation was performed, and the experimental participants were instructed to look at each menu item being highlight. Hereinafter, the three experimental conditions will be respectively described.

(1) The chewing-selection condition: a condition where an intentional chewing motion is to be made upon highlighting of an item meant to be selected. More specifically, it is a condition of imposing a task of making an intentional chewing motion upon highlighting, with a chewing method as instructed.

As used in the present specification, an “intentional chewing” means, after taking a state where the teeth lightly meet (i.e., a state where the teeth of the upper jaw are in contact with the teeth of the lower jaw), transitioning to a state where a pressure of biting force is applied, for example. As the chewing method under the chewing-selection condition, the inventors gave an instruction that, immediately after an item meant to be selected is highlighted, a chew should be made for a short time following a state where the teeth lightly meet.

(2) The speaking condition: a condition of imposing a task of continuously speaking while looking at the menu item being highlighted.

(3) The eating condition: a condition of imposing a task of eating a meal while looking at the menu item being highlighted.

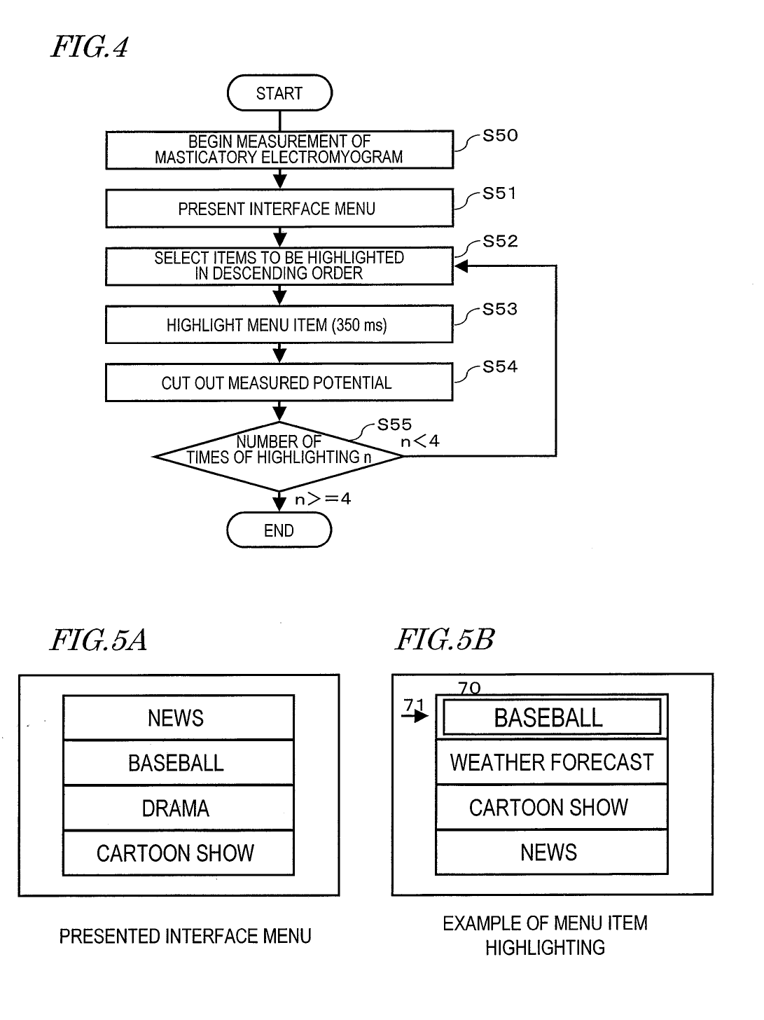

With reference to FIG. 4, a flow of a device which performs menu presentation with highlights and potential measurement during the experiment will be described. The flow of the device side is similar among the aforementioned three conditions.

Step S50 is a step of beginning measurement of the potential (masticatory electromyogram) of a test subject.

step S51 is a step of presenting four menu items of a highlight-type menu, thus indicating to the user what kinds of menu items there are. FIG. 5A is a simplified diagram of the menu items which were actually presented to the test subjects. In the inventors' experiment, each presentation was made for 2 seconds. Note that step S51 also has an effect of stabilizing the measured potential before beginning menu item highlighting and reducing noises such as an electro-oculographic potential.

Step S52 is a step of selecting a menu item to be next highlighted in descending order.

step S53 is a step of highlighting the menu item selected at step S52 for 350 ms. FIG. 5B is a diagram showing an example of highlighting. As shown in FIG. 5B, the menu item itself may be highlighted, or an indication with an arrow or the like may be made instead of highlighting, or simultaneously with highlighting. Note that FIGS. 5A and 5B are not interrelated; each represents an example in its own right.

Step S54 is a step of cutting out a potential waveform over 100 ms before highlighting (hereinafter denoted as “−100 ms”) and 400 ms after highlighting, based on the point of highlighting a menu item at step S53 as 0 ms. The potential waveform having been cut out is subjected to a baseline correction based on an average potential at 100 ms before highlighting.

Step S55 is a branching based on the number n of times of highlighting the menu item. If the number of times of highlighting is smaller than the number of menu items, control proceeds to step S52, and highlighting is continued until every menu item has been highlighted once. It is presumable that no repetitive highlighting is required because the masticatory electromyogram has a strong signal intensity.

Through step S50 to step S55 above, four potential waveforms based on the highlighting as a starting point can be recorded, each menu item having been highlighted once.

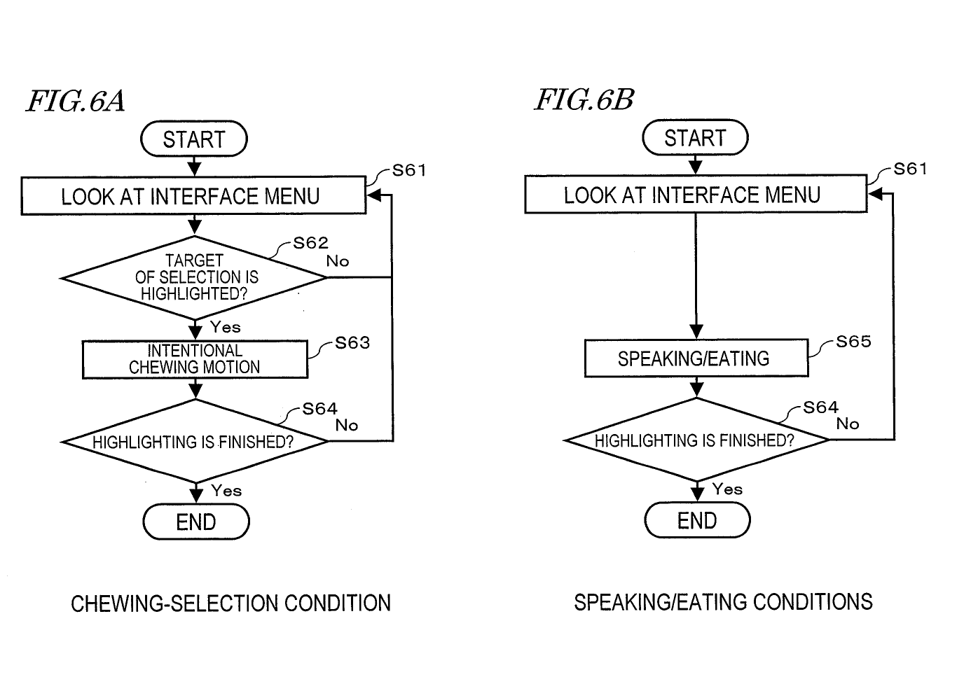

Next, with reference to FIGS. 6A and 6B, a flow of the participant side of the experiment will be described. FIG. 6A is a diagram showing a flow of the chewing-selection condition on the participant side.

Step S61 is a step of looking at a menu which is presented at step S51 in FIG. 4. The test subject is instructed in advance to select a menu item, and moves his or her gaze to a menu item meant to be selected at this point. The instruction of an item meant to be selected corresponds to a device operation which a user wishes to realize when actually using a chewing interface.

Step S62 is a branching based on whether the menu item meant to be selected is highlighted or not while looking at the menu item that is presented through steps S52 to S55 in FIG. 4 being highlighted. If Yes at step S62, control proceeds to step S63; if No, control proceeds to step S61.

Step S63 is a step of making a short-time chewing motion immediately after highlighting if the menu item meant to be selected is highlighted at step S61. The chewing motion is made by a method of “chew for a short time following a state where the teeth lightly meet”, as instructed in advance.

Step S64 is a branching based on whether highlighting has been finished or not. If Yes at step S64, control proceeds to END; if No, control proceeds to step S61.

FIG. 6B is a diagram showing a flow of the speaking condition/eating condition on the participant side. Any step where the same process as in the selecting condition shown in FIG. 6A is denoted by the same reference numeral, and the description thereof is omitted.

A difference from the chewing-selection condition is that step S65 is performed instead of step S62 and step S63 for issuing a masticatory electromyogram for menu selection.

Step S65 is a step of doing an act as instructed in advance, i.e., continuously speaking under the speaking condition or continuously eating a meal under the eating condition.

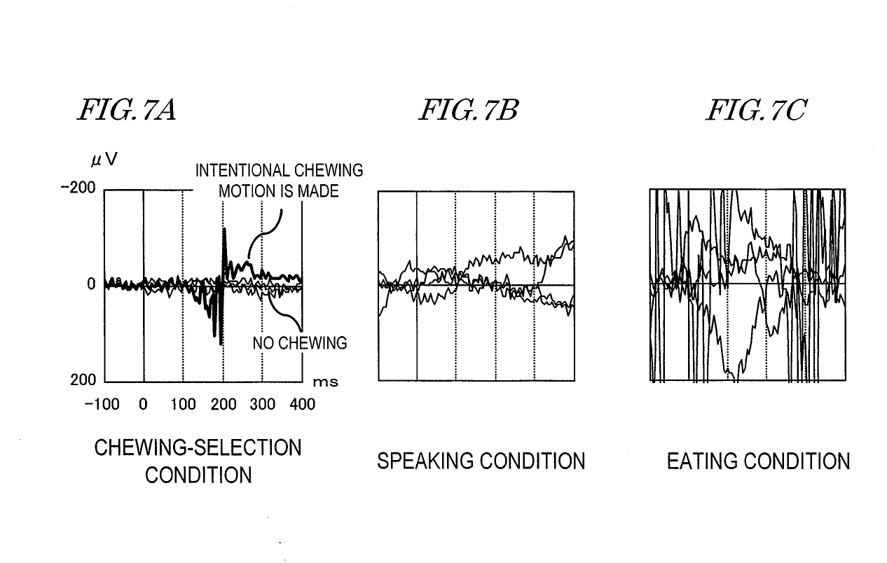

FIGS. 7A to 7C show exemplary experimental results. FIGS. 7A to 7C show potential waveforms obtained by cutting out potential changes from −100 ms to 400 ms based on the highlighting as a starting point, where the horizontal axis represents time in units of ms and the vertical axis represents potential in units of μV, shown at the same scale. The scale is shown only in FIG. 7A.

FIG. 7A illustrates results under the chewing-selection condition, showing (thick solid line) a potential waveform of the case where an intentional chewing motion was made upon highlighting of an item meant to be selected, and (thin solid line) a potential waveform of the case where chewing was not made upon highlighting of an item not meant to be selected. It can be seen that, when a chewing motion was made, a positive potential with a high amplitude and a high frequency appears at approximately 200 ms based on the highlighting of the item meant to be selected as a starting point.

FIG. 7B illustrates results under the speaking condition, showing potential waveforms while continuously speaking irrespective of highlighting of menu items. Since there is no highlight-to-highlight difference in the condition, the waveforms with respect to all highlights are shown by thin solid lines. It can be seen that each potential waveform has a high amplitude but mild fluctuations.

FIG. 7C illustrates results under the eating condition, showing potential waveforms while eating a meal irrespective of highlighting of menu items. Since there is no highlight-to-highlight difference in the condition, the waveforms with respect to all highlights are shown by thin solid lines. It can be seen that high-amplitude and high-frequency potential changes and high-amplitude and low-frequency potential changes are diversely present.

Hereinafter, it will be described as to how use of the characteristic amounts which the inventors employ for distinction allows an intentionally-issued masticatory electromyogram under the chewing-selection condition to be distinguished from: (1) the potential waveform of a masticatory electromyogram when chewing is not performed under the chewing-selection condition; and (2) the potential waveform of a masticatory electromyogram which occurs due to speaking or eating in daily life.

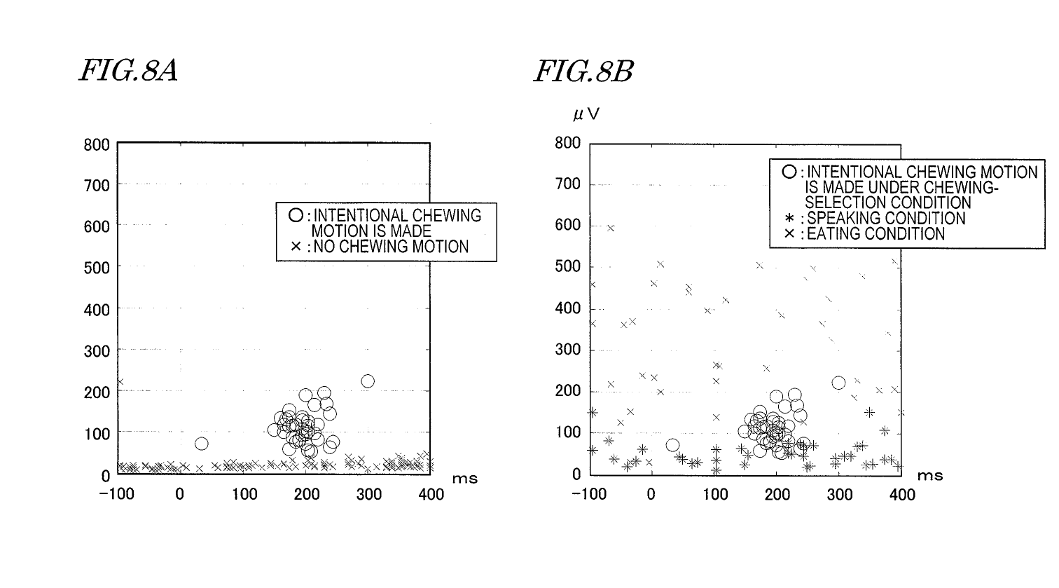

FIG. 8A is a diagram showing potential waveforms under the chewing-selection condition, which are cut out for respective highlights that are accompanied or not accompanied by an intentional chewing motion, plotted by using latency and maximum amplitude as characteristic amounts. As used herein, “latency” refers to a point in time at which the maximum amplitude occurs (i.e., an amount of time in which the maximum amplitude occurs), based on the point of highlighting a menu item as a starting point. A point at which a menu item is highlighted is shown on the horizontal axis of FIG. 8A, in units of ms. The vertical axis represents maximum amplitude in units of μV. The instances where an intentional chewing was made are plotted with ◯ symbols (40), whereas the instances where a chewing was not made are plotted with X symbols (80).

From FIG. 8A, it can be seen that a masticatory electromyogram which occurs due to a chewing motion immediately after menu item highlighting has a small variance in its maximum amplitude and latency. The average values±variances of the maximum amplitude and latency of the masticatory electromyogram were, respectively, 104.3±32.9 (μV) and 201.3±37.8 (ms). Thus, it can be said that an intentional masticatory electromyogram with respect to highlighting of an item meant to be selected will characteristically appear with a latency in the neighborhood of approximately 200 ms and a maximum amplitude in the neighborhood of about 100 μV. This fact was not known before the inventors' experiment under the condition of presenting a highlight-type menu and analysis of the characteristic features of an intentionally issued masticatory electromyogram through cutting out the potential waveform based on the highlighting of a menu item as a starting point, and therefore should be considered as the inventors' own finding.

Moreover, FIG. 8A suggests that presence/absence of an intentional chewing is distinguishable based on maximum amplitude alone. The maximum amplitude when no chewing was made was 23.4±18.1 (mean±standard deviation). As a result of performing a two-tailed t test on the maximum amplitude, a significant difference was found such that p<0.001. For example, performing a distinction by setting the threshold value to 50 μV will result in a distinction ratio of 97.5%. Thus, it can be said that a masticatory electromyogram for an intentional chewing motion immediately after menu item highlighting is distinguishable from that which is obtained when no chewing is made.

FIG. 8B is a diagram showing masticatory electromyograms when an intentional chewing is made under the chewing-selection condition and masticatory electromyograms under the speaking and eating conditions, plotted by using latency and maximum amplitude as characteristic amounts. The horizontal axis of FIG. 8B represents latency (ms), whereas the vertical axis represents maximum amplitude (μV). The instances where an intentional chewing was made are indicated with ◯ symbols; the instances under the speaking condition are indicated with * symbols; and the instances under the eating condition are indicated with X symbols. Note that the plots where an intentional chewing was made (◯ symbols) are the same data as those in FIG. 8A.

The maximum amplitudes under the speaking condition/eating condition were 43.7±30.0 and 316.2±144.4 (mean±standard deviation), respectively. If the maximum amplitude is subjected to threshold processing based on e.g. 50 μV similarly to the aforementioned distinction concerning presence/absence or chewing, 30% of the speaking condition and 95% of the eating condition will be misdetected, and therefore it is difficult to realize a highly accurate distinction by using maximum amplitude alone.

Therefore, based on the aforementioned finding that a masticatory electromyogram under the chewing-selection condition has little variance in its maximum amplitude and latency, we attempted a distinction by introducing latency, as a notion which uniquely pertains to the highlight-type menu presentation condition. For example, if the range with a maximum amplitude and latency of 50 to 150 μV and 150 to 200 ms is to be detected as a masticatory electromyogram with respect to highlighting of an item meant to be selected, then 85% of a masticatory electromyogram under the chewing-selection condition will be correctly detected, while the rate of misdetections of the speaking condition/eating condition will only be 12.5% and 2.5%, respectively. Thus, by applying a range limitation based on maximum amplitude and latency, an intentional masticatory electromyogram with respect to highlighting of an item meant to be selected can be highly accurately detected even in a situation where masticatory electromyograms of daily life may occur. As a result, there is realized a chewing interface which will have few malfunctions even during conversation or while eating, for example.

Each of the interface systems described in connection with Embodiments below detects a masticatory electromyogram which appears due to an intentional chewing motion immediately after highlighting, by relying on maximum amplitude and latency as indices, thus realizing an interface system which enables a rapid menu selection even in a situation where masticatory electromyograms of daily life may occur, e.g., during conversation or while eating, without having to repeat any special chewing motion or highlighting. This is based on the characteristic features of a masticatory electromyogram that is intentionally issued upon highlighting, which the inventors have found through experimentation.

### Embodiment 1

Hereinafter, an outline of an interface system which performs highlight-type menu presentation will be described first. Thereafter, the construction and operation of an interface system of the present embodiment including a masticatory electromyogram detector will be described.

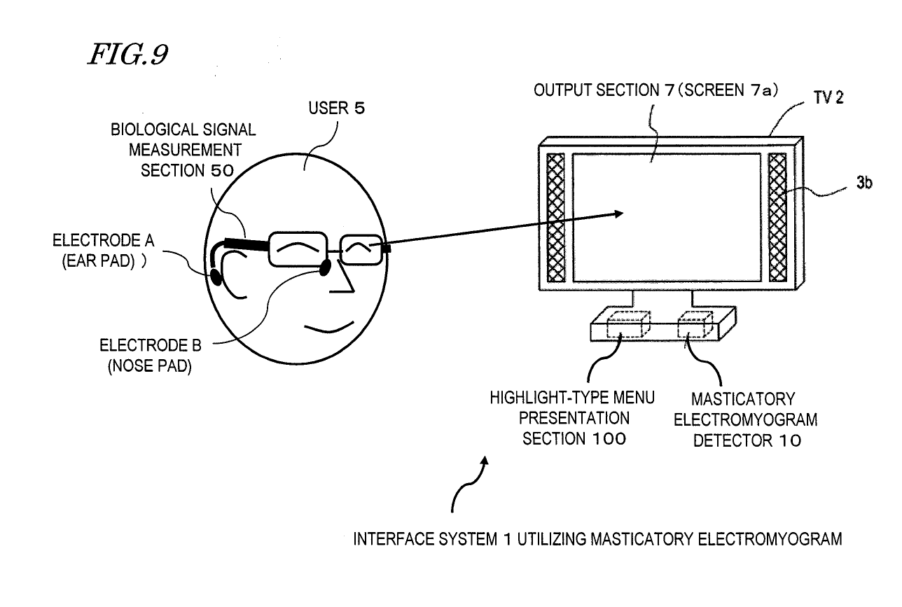

FIG. 9 shows a construction and environment of use for an interface system 1 utilizing a masticatory electromyogram (hereinafter simply referred to as the “interface system 1”). The interface system 1 is exemplified so as to correspond to a system construction according to Embodiment 1 which will be described later.

The interface system 1 is a system for providing an interface for manipulating a TV 2, by utilizing an electrical biological signal (masticatory electromyogram) which is measured in the neighborhood of the head of a user 5 to select a menu item that is presented on an output section 7 by a highlight-type menu presentation section 100. A potential difference between an electrode A which is attached to the nasion (i.e., the site which will be in contact with a nose pad of a pair of glasses) of the user 5 and an electrode B which is attached to a mastoid (i.e., the site which will be in contact with an ear pad of a pair of glasses) of the user 5 is acquired by a biological signal measurement section 50, and is transmitted in a wireless or wired manner to a masticatory electromyogram detector 10. The masticatory electromyogram detector 10, which is internalized in the TV 2, utilizes the transmitted potential change to detect an intention of menu selection of the user, and performs processing such as switching of channels.

FIG. 9 illustrates an example where the TV 2, as a separate element, is manipulated with a glasses-type manipulating device (biological signal measurement section 50). However, when a head-mount display (HMD) is contemplated, for example, all constituent elements, i.e., the output section 7, the highlight-type menu presentation section 100, and the masticatory electromyogram detector 10 may be accommodated within the HMD. In the case of wearing an HMD, too, the nasion and the mastoid are sites which will naturally come into contact with the user 5.

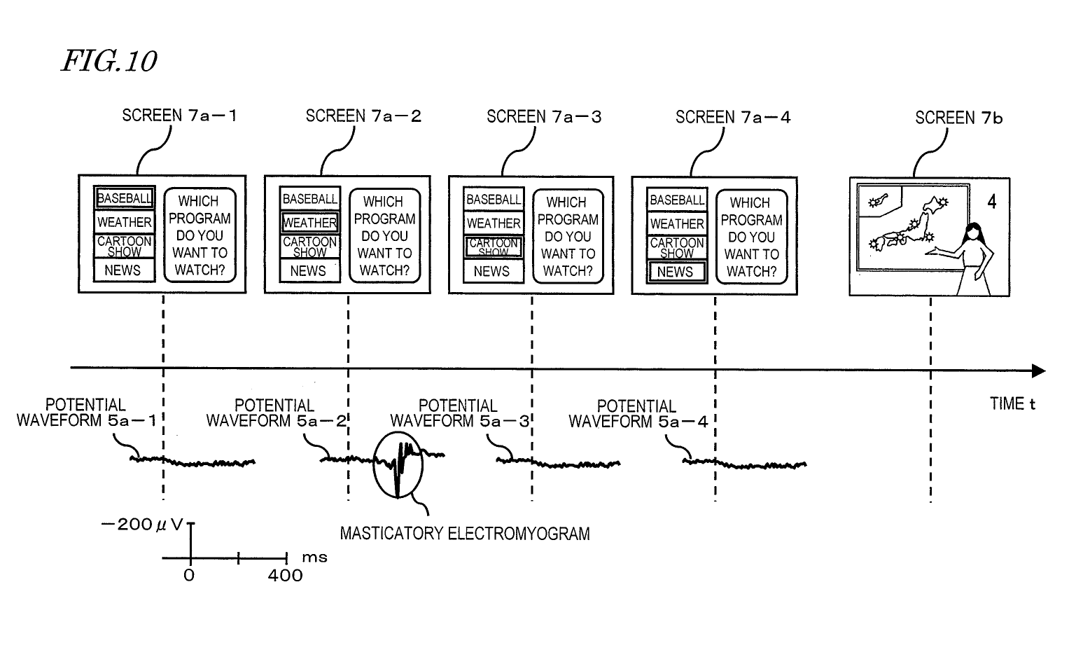

FIG. 10 illustrates an example where the TV 2 is manipulated in the interface system 1 by the user 5 who selects and watches a program which he or she wishes to view.

In the upper part of FIG. 10, screens 7a-1 to 7a-4 respectively illustrate examples of a menu which the highlight-type menu presentation section 100 presents to the user via the screen 7a of the TV 2. FIG. 10 illustrates how four menu items of “baseball”, “weather”, “cartoon show”, and “news” may be highlighted, where the menu items “baseball” and “weather forecast” are highlighted in the screen 7a-1 and the screen 7a-2, and “cartoon show” and “news” in the screen 7a-3 and the screen 7a-4.

By highlighting the menu items, it becomes possible to cut out a potential change in the masticatory electromyogram detector 10, based on the point of highlighting each menu item as a starting point. In the lower part of FIG. 10, potential waveforms 5a-1 to 5a-4 schematically represent potential waveforms which are cut out in the masticatory electromyogram detector 10 based on the highlighting of each menu item as a starting point.

Now, it is assumed that the user wishes to watch the “weather” channel, and makes an intentional chewing motion immediately after “weather” is highlighted. The potential waveform 5a-2 indicates, in this situation, how an intentionally issued masticatory electromyogram may appear in the neighborhood of approximately 200 ms since “weather forecast” is highlighted. The screen 7b in the upper part of FIG. 10 illustrates the channel having been switched to “weather forecast” because of a masticatory electromyogram intentionally issued upon highlighting of “weather forecast” being detected in the masticatory electromyogram detector 10 (5a-2).

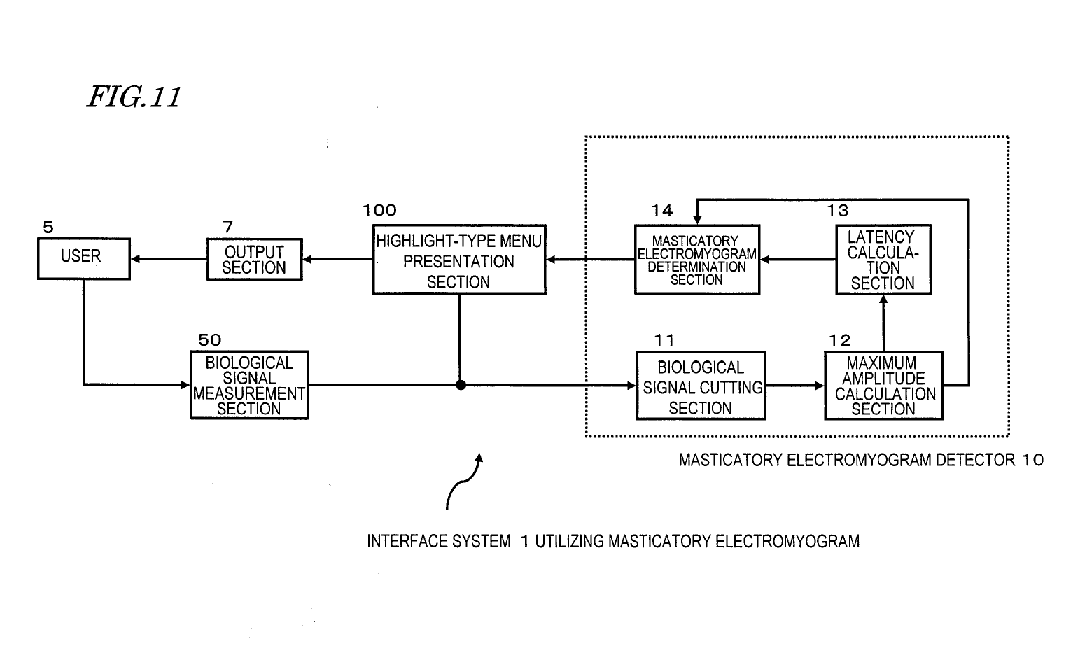

FIG. 11 shows a functional block construction of the interface system 1 of the present embodiment. The interface system 1 includes the output section 7, the masticatory electromyogram detector 10, the biological signal measurement section 50, and the highlight-type menu presentation section 100. FIG. 11 also shows detailed functional blocks of the masticatory electromyogram detector 10. The user 5 block is illustrated for convenience of explanation. The output section 7 presents a screen on which a menu and the like are presented to the user 5.

The user 5 pays attention to whether menu items concerning device manipulations which are presented by the highlight-type menu presentation section 100 on the output section 7 are highlighted or not, and merely makes an intentional chewing motion upon highlighting of an item meant to be selected, without making any manipulation input via a button or the like. The interface system 1 detects the chewing motion of the user, identifies a menu item to which the chewing motion was directed, and operates a device in accordance with the menu item which has been selected via the highlight-type menu presentation section 100. For example, this “device” may be a TV corresponding to the output section 7, and the “operation” may be a channel switch operation, although such is only an example. It may be any device other than a TV, e.g., a videorecording apparatus or a DVD player (not shown).

The masticatory electromyogram detector 10 is connected to the biological signal measurement section 50 and the highlight-type menu presentation section 100 in a wireless or wired manner, and performs transmission and reception of signals. Although FIG. 11 illustrates the biological signal measurement section 50 and the highlight-type menu presentation section 100 as separate entities from the masticatory electromyogram detector 10, this is only an example. A part or whole of the biological signal measurement section 50 and the highlight-type menu presentation section 100 may be provided within the masticatory electromyogram detector 10.

The biological signal measurement section 50 is an electromyograph for detecting a biological signal of the user 5, and measures an electromyogram which occurs from mastication or the like as a biological signal. By using a glasses-type device as shown in FIG. 9 as the biological signal measurement section 50, a potential difference between a nose pad and an ear pad may be measured. It is assumed that the user 5 is already wearing the biological signal measurement section 50.

As a result, the biological signal measurement section 50 is able to measure a potential change in the neighborhood of the face of the user 5. The measured potential change of the user 5 is sampled so as to be computer-processable, and is sent to the masticatory electromyogram detector 10 in real time. Note that, in order to reduce the influence of noise on commercial power which may be mixed in the measured potential, the potential to be measured in the biological signal measurement section 50 is subjected to band-pass filtering from e.g. 0.1 Hz to 30 Hz in advance, and to baseline correction with respect to an average potential at e.g. 100 ms before highlighting of menu items.

The highlight-type menu presentation section 100 highlights menu items concerning device manipulations at an interval of 350 ms, for example. In order to be able to identify which highlight a masticatory electromyogram is directed to, the highlighting interval needs to be set equal to or greater than the variance of the latency of masticatory electromyograms. Since the variance of latency in the aforementioned experimental results was 37.8 (ms), the highlighting interval may be set to 100 ms, for example. The highlight-type menu presentation section 100 gives an instruction to control a device operation in accordance with the result of distinction by the masticatory electromyogram detector 10. Assuming that the device to be controlled by utilizing the highlight-type menu presentation section 100 is the TV 2 shown in FIG. 9, for example, the menu is presented to the user 5 via the output section 7 (screen 7a).

Next, the detailed construction of the masticatory electromyogram detector 10 according to the present embodiment will be described. A main characteristic feature of the present concerns the construction and operation of the masticatory electromyogram detector 10.

The masticatory electromyogram detector 10 includes a biological signal cutting section 11, a maximum amplitude calculation section 12, a latency calculation section 13, and a masticatory electromyogram determination section 14.

Based on a timing of menu item highlighting which is sent from the highlight-type menu presentation section 100 as a starting point, the biological signal cutting section 11 cuts out a potential waveform of the masticatory electromyogram which is sent from the biological signal measurement section 50 in real time, and subjects the potential waveform having been cut to baseline correction. The time slot in which to cut out the potential waveform may be −100 ms to 400 ms, or −300 ms to 300 ms, so as to include the time slot from 150 ms to 300 ms after highlighting, where the timing of menu item highlighting is defined as 0 ms. The time period may be chosen so that no menu item will be highlighted a plurality of times, given the highlighting interval. Moreover, the baseline correction may be performed by a method of subtracting an average potential over 100 ms before menu item highlighting from the total potential, or by using an average potential over an arbitrary time period. After the waveform cutting and the baseline correction are completed, the biological signal cutting section 11 sends the potential waveform to the maximum amplitude calculation section 12.

The maximum amplitude calculation section 12 determines a maximum amplitude of the potential waveform of the masticatory electromyogram sent from the biological signal cutting section 11, and sends this information to the masticatory electromyogram determination section 14. The maximum amplitude of the potential waveform of the masticatory electromyogram is a maximum value of potential relative to 0 μV, for example. Moreover, the maximum amplitude calculation section 12 sends the potential signal of the masticatory electromyogram to the latency calculation section 13. Note that the waveform may be sent to the latency calculation section 13 only when the maximum amplitude is equal to or greater than a threshold value. In this case, the threshold value may be e.g. 100 μV, or a value may be set for each user.

The latency calculation section 13 determines the latency (i.e., an amount of time in which the potential waveform arrives at the maximum amplitude, based on the point of menu item highlighting as 0 ms) of the potential waveform sent from the maximum amplitude calculation section 12, and sends the information of the determined latency to the masticatory electromyogram determination section 14.

Note that the potential waveform which is to be processed by the maximum amplitude calculation section 12 and the latency calculation section 13 is a waveform which is obtained by being cut out by the biological signal cutting section 11. Therefore, the start point of the received waveform corresponds to the timing of menu item highlighting. Therefore, the maximum amplitude calculation section 12 and the latency calculation section 13 are able to determine the aforementioned maximum amplitude and latency by ascertaining a maximum amplitude of the received waveform and the amount of time up to then.

Based on the maximum amplitude and latency sent from the maximum amplitude calculation section 12 and the latency calculation section 13, the masticatory electromyogram determination section 14 determines presence or absence of a masticatory electromyogram which is intentionally issued upon highlighting of a menu item, and sends the result to the highlight-type menu presentation section. As the determination criterion, any instance where the maximum amplitude and latency are in the ranges of 100±50 μV and 200±50 ms may be detected to indicate that the user 5 made an intentional chewing motion immediately after menu item highlighting, for example. Alternatively, average values and variances of the maximum amplitude and latency of each user may be measured, and a range of “average value±standard deviation” may be set for each parameter.

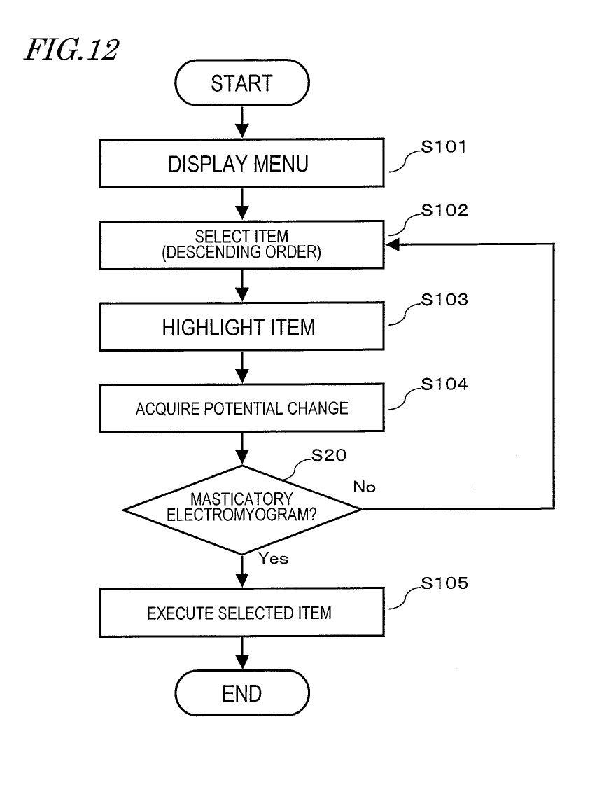

Next, with reference to the flowchart of FIG. 12, the overall procedure of processing performed by the interface system 1 shown in FIG. 11 will be described.

FIG. 12 shows a processing procedure of the interface system 1 for determining whether a user made a short-time intentional chewing motion immediately after menu item highlighting or not.

At step S101, the highlight-type menu presentation section 100 presents a manipulation menu of e.g. four menu items (for example, FIG. 5A).

At step S102, the highlight-type menu presentation section 100 consecutively selects a menu item to be next highlighted, one by one from the top.

At step S103, the menu item selected at step S102 is highlighted. The highlighting interval is chosen so as to be greater than the variance of the latency of masticatory electromyograms and so that the user 5 is able to recognize highlighting, and may be e.g. 100 ms.

At step S104, the biological signal measurement section 50 measures a potential change (masticatory electromyogram) of the user 5.

At step S20, the masticatory electromyogram detector 10 determines whether a masticatory electromyogram which is intentionally issued upon highlighting of a menu item is contained in the potential waveform measured at step S104. The detailed processing procedure of step S20 will be described later.

If Yes at step S20, control proceeds to step S105; if No, control returns to step S102 to select a next menu item.

At step S106, the highlight-type menu presentation section 100 executes a process corresponding to the menu item selected at step S20. As a result, this menu item is selected and executed.

With the interface system 1 as such, even during conversation or while eating, a rapid menu selection can be realized without having to repeat any special chewing motion or highlighting. Thus, a significantly improved device manipulability is obtained.

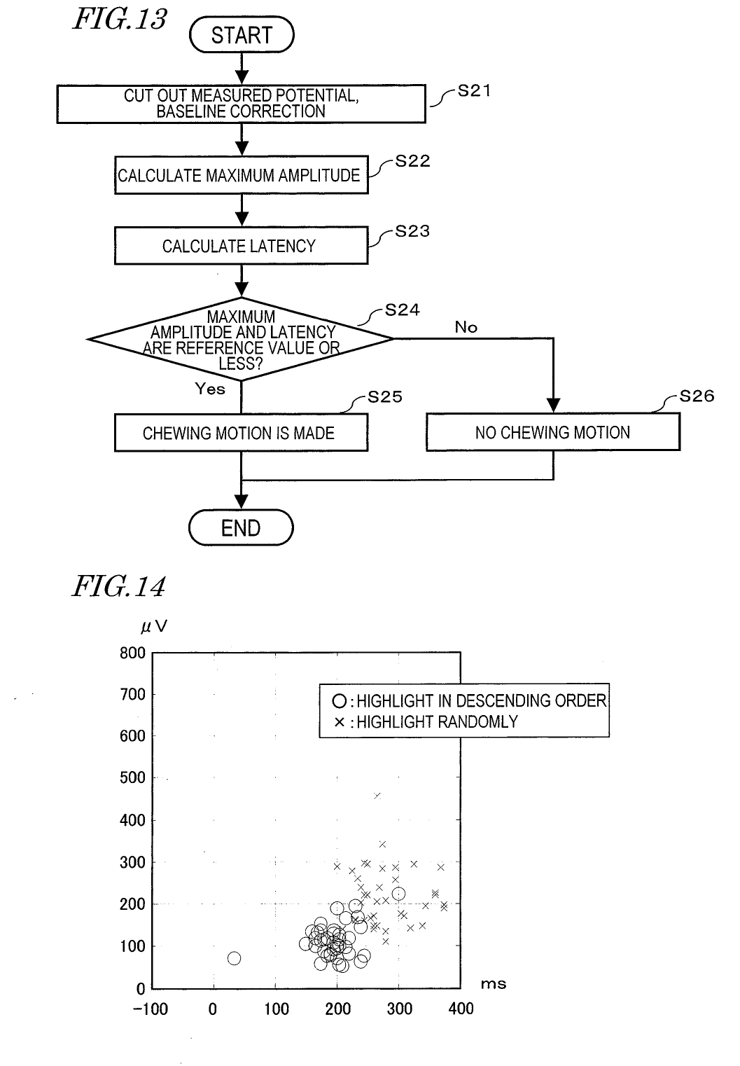

FIG. 13 shows a detailed procedure of determining whether or not the user 5 made an intentional chewing motion immediately after menu item highlighting, which is realized by the biological signal cutting section 11, the maximum amplitude calculation section 12, the latency calculation section 13, and the masticatory electromyogram determination section 14 composing the masticatory electromyogram detector 10.

That is, at step S21 in FIG. 13, from a potential change at the nasion and a mastoid of the user 5 as measured by the biological signal measurement section 50, the biological signal cutting section 11 cuts out a potential waveform of the masticatory electromyogram, based on the timing of highlighting the menu item in the highlight-type menu presentation section 100 as a starting point, and subjects it to baseline correction. The time slot in which to cut out the potential waveform may be −100 ms to 400 ms, or −300 ms to 300 ms, so as to include the time slot from 150 ms to 300 ms after highlighting, where the timing of menu item highlighting is defined as 0 ms. Moreover, the baseline correction may be performed by a method of subtracting an average potential over 100 ms before menu item highlighting from the total potential, or by using an average potential over an arbitrary time period. The biological signal cutting section 11 sends the potential waveform after baseline correction to the maximum amplitude calculation section 12.

At step S22, the maximum amplitude calculation section 12 receives a potential waveform from the biological signal cutting section 11 and determines a maximum value of amplitude, and sends the potential waveform to the latency calculation section 13. On the other hand, it sends the determined maximum amplitude information to the masticatory electromyogram determination section 14.

At step S23, the latency calculation section 13 determines the latency (i.e., an amount of time in which the potential waveform arrives at the maximum amplitude, based on the point of menu item highlighting as 0 ms) of the potential waveform, and sends the information of the determined latency to the masticatory electromyogram determination section 14.

At step S24, based on the maximum amplitude and latency sent from the maximum amplitude calculation section 12 and the latency calculation section 13, the masticatory electromyogram determination section 14 determines presence or absence of a masticatory electromyogram which is intentionally issued upon highlighting of a menu item. As the determination criterion, any instance where the maximum amplitude and latency are in the ranges of 100±50 μV and 200±50 ms may be detected, for example. Alternatively, average values and variances of the maximum amplitude and latency may be measured, and a range of “average value±standard deviation” may be set for each parameter. If Yes at step S24, control proceeds to step S25; if No, control proceeds to step S26.

At step S25, the masticatory electromyogram determination section 14 determines that the user 5 made an intentional chewing motion immediately after menu item highlighting.

At step S25, the masticatory electromyogram determination section 14 determines that the user 5 did not make a chewing motion immediately after menu item highlighting.

Through such processing, a chewing motion which a user made for menu selection immediately after highlighting can be detected.

With the interface system 1 of the present embodiment, by using as characteristic amounts the maximum amplitude and latency of a potential waveform based on the highlighting of a menu item as a starting point, a masticatory electromyogram occurring due to an intentional chewing motion immediately after highlighting can be detected. As a result, there is realized an interface which is easy to use, permits rapid menu selection, and will have few malfunctions during conversation or while eating. Specifically, there is realized an interface which, in the case of talking on a mobile phone and simultaneously making a menu selection within another function (e.g. an Internet function) as multi-tasking, has a low possibility of allowing an incorrect menu selection to occur because of a masticatory electromyogram that appears due to the conversation. Moreover, there is realized an interface which will have few malfunctions because of a masticatory electromyogram that appears due to eating when a highlight-type menu concerning a recommended program is automatically presented while watching TV and simultaneously eating a meal, for example.

### Embodiment 2

In the interface system 1 of Embodiment 1, the highlight-type menu presentation section 100 highlights menu items consecutively from top to bottom, and detects an intentional masticatory electromyogram which stably appears in the neighborhood of approximately 200 ms based on the highlighting as a starting point, by relying on maximum amplitude and latency as indices. Thus, an interface is realized which permits rapid menu selection and which will have few malfunctions during conversation or while eating.

In addition to the above-described experiment, the inventors have performed an additional experiment (random experiment) under a condition of highlighting menu items in random order, thereby finding that the maximum amplitude and latency of an intentionally issued masticatory electromyogram will change in the case of highlighting menu items in random order.

Therefore, in the interface system of the present embodiment, in detecting an intentionally issued masticatory electromyogram, the detection target ranges for maximum amplitude and latency are switched in accordance with the order of menu item highlighting. As a result, even when menu items are highlighted in random order, it becomes possible to highly accurately detect an intentional masticatory electromyogram occurring due to a chewing motion immediately after highlighting. By performing highlighting in random order, there emerges a possibility that a menu item to be selected which happens to be at a lower position on the list may be highlighted earlier, thus leading to an advantage of reducing the manipulating time. Therefore, a random highlighting order will be effective especially when the menu items which are frequently selected by the user are known to the device in advance.

First, the random experiment will be described. In the random experiment, step S52 in FIG. 4 was modified so that the order of menu item highlighting was random. Otherwise, the experimental settings were similar to those under the chewing-selection condition in the aforementioned experiment (descending-order experiment), and therefore detailed descriptions of the experiment will be omitted.

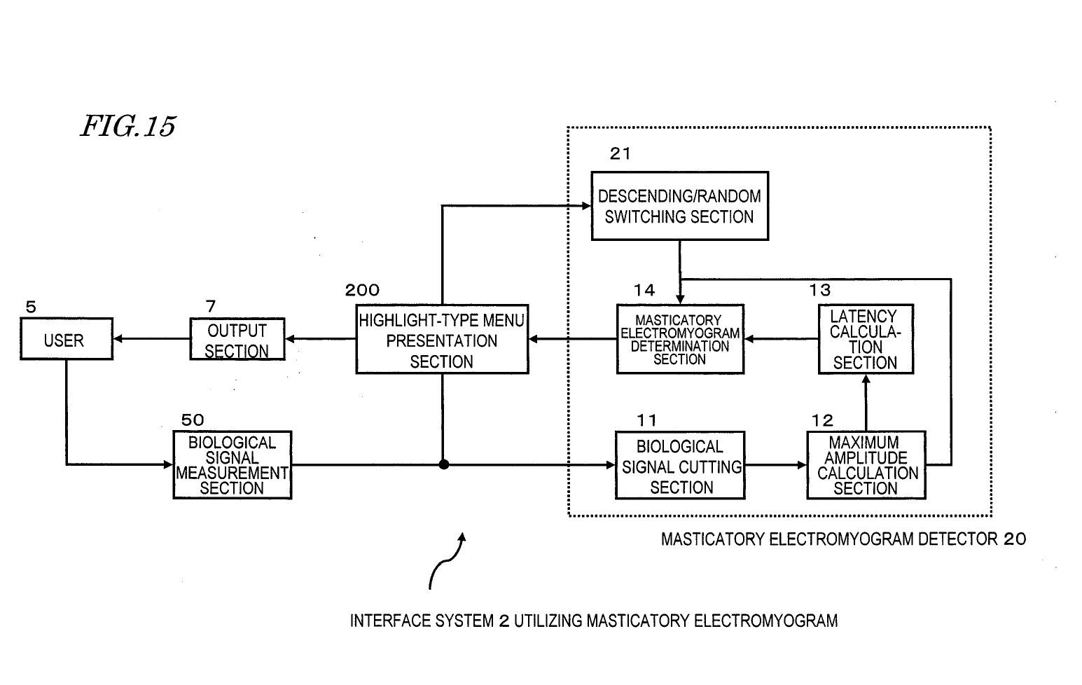

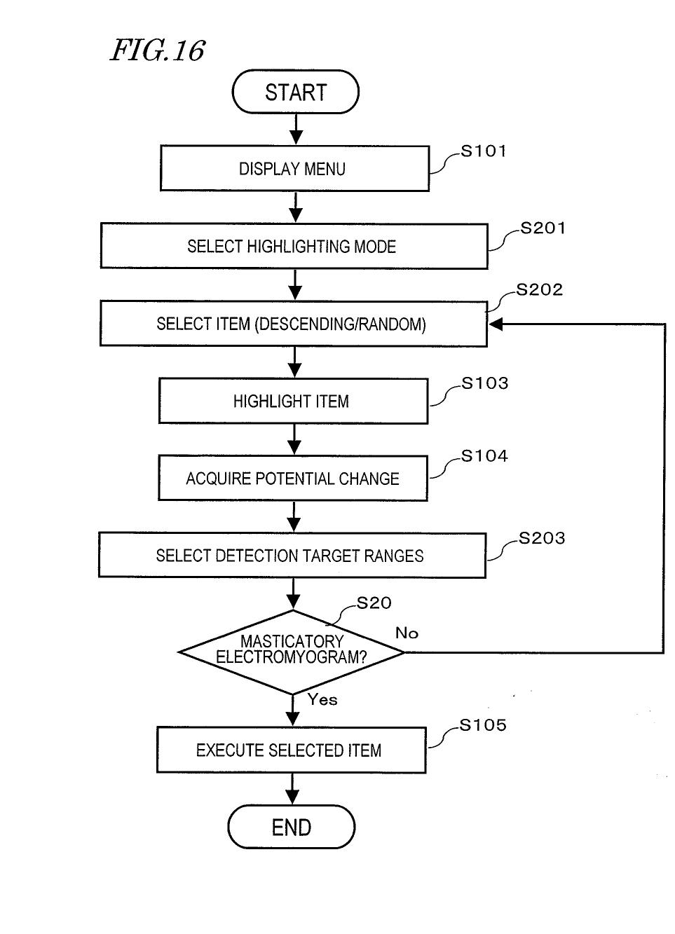

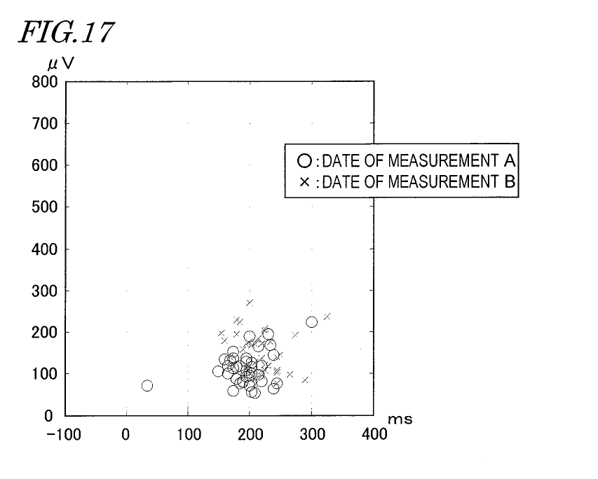

FIG. 14 shows results under the chewing-selection condition in the random experiment and the descending-order experiment, plotted by using latency and maximum amplitude as characteristic amounts. The horizontal axis represents time in units of ms (where the point of menu item highlighting is defined as 0 ms), whereas the vertical axis represents maximum amplitude in units of μV. The instances where an intentional chewing was made in the descending-order experiment are plotted with ◯ symbols (40), whereas the instances where an intentional chewing was made in the random experiment are plotted with X symbols (40). It can be seen that, in the random experiment, the maximum amplitude and latency of the masticatory electromyogram of immediately after highlighting are both shifted in the plus direction relative to the results of the descending-order experiment. The average value±variance of maximum amplitude and latency in the random experiment were 213.0±74.5 (μV) and 282.9±50.7 (ms), respectively, indicative that the average value and variance value of maximum amplitude and latency were both increased from those under the descending order condition. Note that at test indicated significantly different maximum amplitude and latency (p<0.05). Thus, it became clear that the characteristic features of an masticatory electromyogram to appear will vary depending on the order of highlighting. A possible reason for the changes in maximum amplitude and latency is that the timing with which an item meant to be selected is highlighted is not predictable in random-order highlighting, thus slowing the response. It can also be seen that, since the maximum amplitude and latency under the random experiment have a larger variance than under the descending order condition, the intentionally issued masticatory electromyogram under the descending order condition provides a higher detection accuracy. Therefore, the method of highlighting the menu items may be switched so that highlighting is performed in descending order if the number of menu items is small (e.g. 10 items or less), and in random order if the number of menu items is large.

It is expected that the rate of misdetecting masticatory electromyograms of daily life will be improved by ensuring that detection target ranges of masticatory electromyogram are ranges of maximum amplitude and latency within which the characteristic features of the masticatory electromyogram under both conditions are encompassed. Moreover, since the order of menu item highlighting is either descending or random, adaptations can be made by switching the detection target ranges for maximum amplitude and latency depending on the order of menu item highlighting.

FIG. 15 shows a functional block construction of an interface system 2 utilizing a masticatory electromyogram according to the present embodiment (hereinafter referred to as “interface system 2”). FIG. 15 also shows detailed functional blocks of a masticatory electromyogram detector 20. The user 5 block is illustrated for convenience of explanation.

Differences of the interface system 2 from the interface system 1 (FIG. 11) are that a highlight-type menu presentation section 200, which is capable of switching the highlighting order between descending/random depending on the mode of menu item highlighting, is included instead of the highlight-type menu presentation section 100, and that a descending/random switching section 21 is newly introduced to the construction of the masticatory electromyogram detector 20. Among the constituent elements of the interface system 2, constituent elements which are identical to those of the interface system 1 will be denoted by like reference numerals, and the descriptions thereof will be omitted.

By switching the mode concerning the order of menu item highlighting, the highlight-type menu presentation section 200 highlights menu items in descending order or randomly. Considering the tendency that the variance of maximum amplitude and latency increases in the random experiment, the highlighting may be switched so as to occur in descending order if the number of menu items is small (e.g. 10 items or less), and in random order if the number of menu items is large.

The descending/random switching section 21 acquires a mode of menu item highlighting from the highlight-type menu presentation section 200, and changes the detection target ranges for masticatory electromyogram that are retained in the masticatory electromyogram determination section 14. For example, the detection target ranges for maximum amplitude and latency may be 100±50 μV and 200±50 ms if the highlighting order is descending, or 200±100 μV and 300±100 ms if it is random.

With such a construction, the detection target ranges for maximum amplitude and latency can be changed depending on the highlighting order, so that a masticatory electromyogram can be detected with a high accuracy, regardless of descending or random highlighting.

Next, with reference to a flowchart of FIG. 16, the overall processing procedure which is performed in the interface system 2 will be described.

FIG. 16 shows a processing procedure of the interface system 2 according to the present embodiment. In FIG. 16, any step where the same process as the process of the interface system 1 (FIG. 12) is performed is denoted by the same reference numeral, and the description thereof is omitted.

At step S201, the highlight-type menu presentation section 200 selects a mode concerning the order of menu item highlighting (descending or random).

At step S202, based on mode selected at step S201, the highlight-type menu presentation section 200 selects a menu item to be next highlighted.

At step S203, the descending/random switching section 21 acquires the mode of highlighting from the highlight-type menu presentation section 200, and changes the detection target ranges for masticatory electromyogram that are retained in the masticatory electromyogram determination section 14. For example, the detection target ranges for maximum amplitude and latency may be 100±50 μV and 200±50 ms if the highlighting order is descending, or 200±100 μV and 300±100 ms if it is random.

Through such processing, the detection target ranges for maximum amplitude and latency can be changed depending on the highlighting order, so that a masticatory electromyogram can be detected with a high accuracy, regardless of descending or random highlighting.

With the interface system 2 of the present embodiment, regardless of whether menu items are highlighted in descending order or randomly, a masticatory electromyogram can be detected with a high accuracy by changing the detection target ranges for maximum amplitude and latency. As a result, an interface which is easy to use, permits rapid menu selection, and will have few malfunctions during conversation or while eating can be realized, especially because of highlighting in random order in the presence of a large number of menu items.

### Embodiment 3

In the interface system 1 of Embodiment 1, the highlight-type menu presentation section 100 highlights menu items consecutively from top to bottom, and detects an intentional masticatory electromyogram which stably appears in the neighborhood of approximately 200 ms based on the highlighting as a starting point, by relying on maximum amplitude and latency as indices. Thus, an interface is realized which permits rapid menu selection and which will have few malfunctions during conversation or while eating.

Although it would be ideal to always maintain malfunctions at a low level, the characteristic features of an intentionally issued masticatory electromyogram may vary due to factors such as not being able to perform the same manner of chewing due to lapse of time, changes occurring in the state in which the electrodes are worn, and so on. This may possibly cause a lower detection accuracy of a masticatory electromyogram.

For example, FIG. 17 is a diagram in which results of performing a descending-order experiment for the same experimental participants on different days, plotted by using latency and maximum amplitude as characteristic amounts. The horizontal axis represents time in units of ms (where the point of menu item highlighting is defined as 0 ms), whereas the vertical axis represents maximum amplitude in units of μV. ◯ symbols (40) and X symbols (40) represent results on different days. The experimental participants were instructed to perform chewing in the same manner on both days. In FIG. 17, it can be seen from a comparison between ◯ symbols and X symbols that they have resulted in different maximum amplitudes, although fluctuations among ◯ symbols and fluctuations among X symbols are small. It can also be seen that there is little change in latency. The maximum amplitude and latency (mean±standard deviation) were 104.3±32.9 (μV) for ◯ symbols, and 201.3±37.8 (ms), 196.1±61.9 (μV) and 220.8±53.7 (ms) for X symbols. Presumable causes for the different maximum amplitudes from day to day are: (1) not being able to perform the same manner of chewing due to lapse of time; and (2) changes occurring in the state in which the electrodes are worn.

In the interface system of the present embodiment, at the first selection after a certain amount of time or more has elapsed, the detection target ranges for maximum amplitude and latency are expanded and an intentionally issued masticatory electromyogram is detected in accordance with the order of menu item highlighting, and new detection target ranges are determined based on the maximum amplitude and latency of that masticatory electromyogram. Thus, changes in the manner of chewing and in the state in which the electrodes are worn are absorbed, thus conserving the detection accuracy of a masticatory electromyogram.

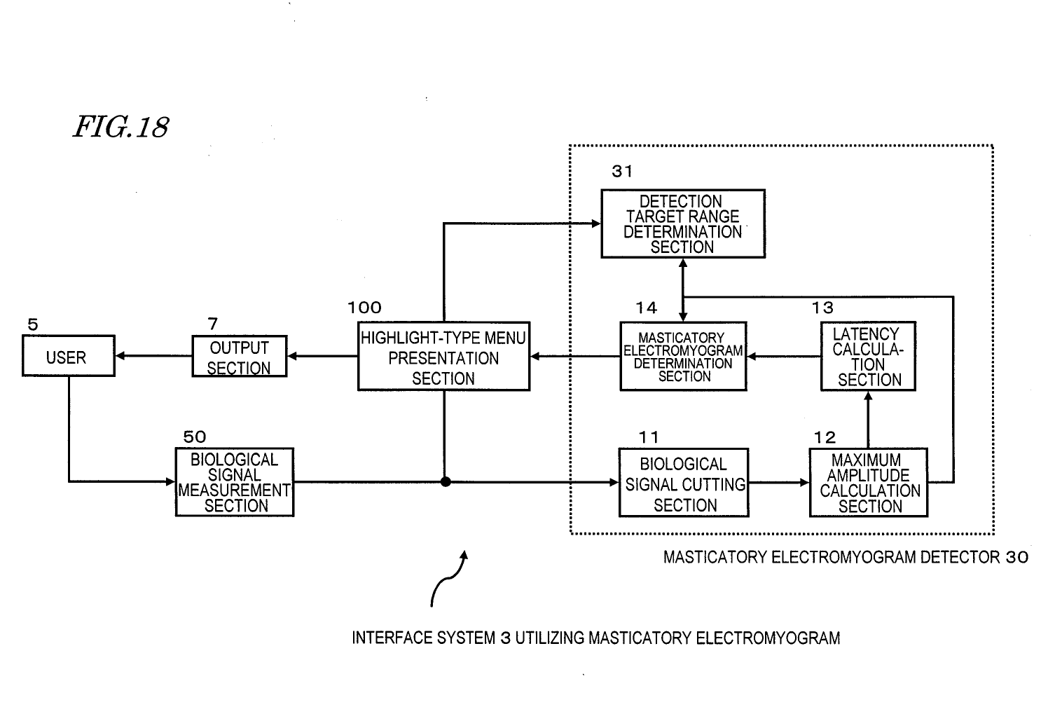

FIG. 18 shows a functional block construction of an interface system 3 utilizing a masticatory electromyogram according to the present embodiment (hereinafter referred to as “interface system 3”). FIG. 18 also shows detailed functional blocks of a masticatory electromyogram detector 30. The user 5 block is illustrated for convenience of explanation.

Differences of the interface system 3 from the interface system 1 (FIG. 11) are that a detection target range determination section 31 is newly introduced to the construction of the masticatory electromyogram detector 30. Among the constituent elements of the interface system 2, constituent elements which are identical to those of the interface system 1 will be denoted by like reference numerals, and the descriptions thereof will be omitted.

The detection target range determination section 31 retains the time which has elapsed since the highlight-type menu presentation section 100 presented a menu to the user 5, and if a certain amount of time or more has elapsed since the previous menu presentation, changes the detection target ranges for masticatory electromyogram retained in the masticatory electromyogram determination section 14 so that the ranges are expanded. The certain amount of time may be 1 hour, or 1 day, for example. As for the detection target ranges, maximum amplitude and latency of 100±75 μV and 200±75 ms may be employed, for example, so long as such changes are in the direction of expanding the ranges. Moreover, when the masticatory electromyogram determination section 14 detects an intentionally issued masticatory electromyogram, the detection target range determination section 31 acquires the maximum amplitude and latency thereof, sets new detection target ranges, and updates the detection target ranges for masticatory electromyogram that are retained in the masticatory electromyogram determination section 14. The new detection target ranges may be determined as “acquired maximum amplitude ±50 μV” and “acquired latency ±50 ms”, for example, or may be determined in accordance with the variance of the maximum amplitude and latency of each user.

With such a construction, changes in the manner of chewing and in the state in which the electrodes are worn are absorbed when a certain amount of time or more has elapsed since the previous menu selection, whereby a masticatory electromyogram can be detected with a high accuracy. Alternatively, after the certain amount of time has elapsed, a menu item highlighting stimulation for calibration purposes may be presented by the highlight-type menu presentation section 100, and the maximum amplitude and latency of an intentional masticatory electromyogram with respect to the menu item highlighting for calibration purposes may be acquired for use as central values for the subsequent menu selections.

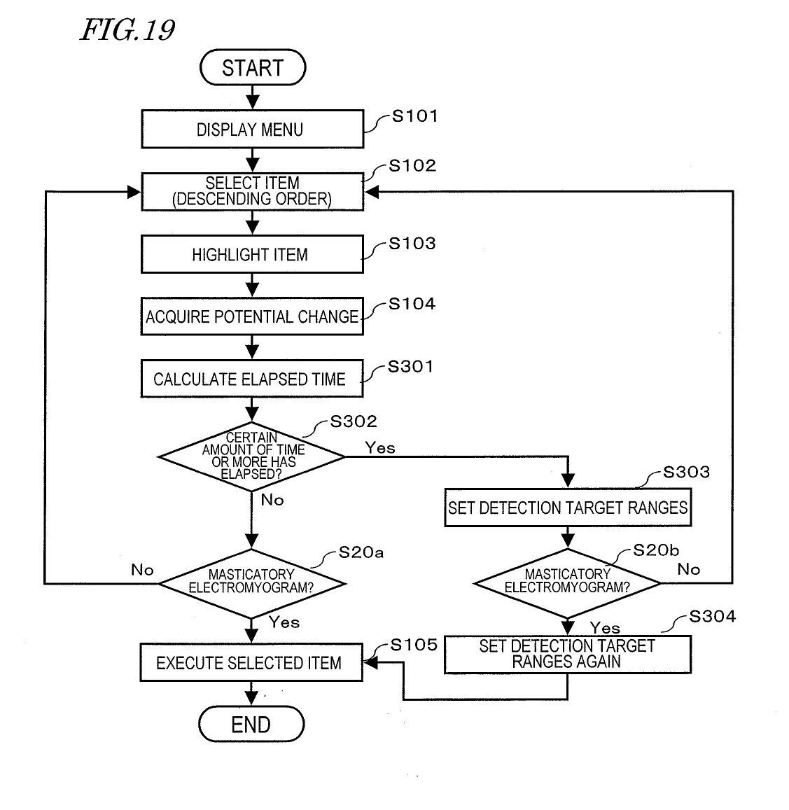

Next, with reference to a flowchart of FIG. 19, the overall processing procedure which is performed by the interface system 3 will be described.

FIG. 19 shows a processing procedure of the interface system 3 of the present embodiment. In FIG. 19, any step where the same process as the process of the interface system 1. (FIG. 12) is performed is denoted by the same reference numeral, and the description thereof is omitted. Note that step S20a and step S20b are the same process as step S20, although directed to different detection target ranges.

At step S301, the detection target range determination section 31 determines an elapsed time since the highlight-type menu presentation section 100 last presented a menu.

At step S302, control of the detection target range determination section 31 branches out depending on whether the elapsed time determined at step S301 is equal to a certain amount or more. If Yes at step S302, control proceeds to step S303; if No, control proceeds to step S20a. The certain amount of time may be 1 hour, or 1 day, for example.

At step S303, the detection target range determination section 31 changes the detection target ranges for masticatory electromyogram retained in the masticatory electromyogram determination section 14 so that the ranges are expanded. The maximum amplitude and latency may be set to be 100±75 μV and 200±75 ms, for example.

At step S304, the detection target range determination section 31 acquires the detected maximum amplitude and latency of the masticatory electromyogram from the masticatory electromyogram determination section 14, sets new detection target ranges, and updates the detection target ranges for masticatory electromyogram that are retained in the masticatory electromyogram determination section 14. The new detection target ranges may be determined as “acquired maximum amplitude ±50 μV” and “acquired latency ±50 ms”, for example, or may be determined in accordance with the variance of the maximum amplitude and latency of each user.

Such processing makes it possible to absorb changes in the manner of chewing and in the state in which the electrodes are worn when a certain amount of time or more has elapsed since the previous menu selection, and thus a masticatory electromyogram can be detected with a high accuracy.

In the interface system of the present embodiment, at the first selection after a certain amount of time or more has elapsed, the detection target ranges for maximum amplitude and latency are expanded and a masticatory electromyogram is detected in accordance with the order of menu item highlighting, and new detection target ranges are determined based on the maximum amplitude and latency of that masticatory electromyogram. Thus, changes in the manner of chewing and in the state in which the electrodes are worn are absorbed, thus conserving the detection accuracy of an intentionally issued masticatory electromyogram. Note that frequent changes are expected in the state in which the electrodes are worn especially in a situation where the user makes a menu selection while moving; in that case, the certain amount of time may be set as short as e.g. 10 minutes in order to perform frequent calibrations.

Although “μV” is employed as a unit of amplitude in the above description, the specific measurement values may differ depending on the device which is used for measurement, due to differences in the amplification ratio and the like. For example, the device which was employed by the inventors as the biological signal measurement section was AP1124 manufactured by DIGITEX LAB. CO., LTD. When a masticatory electromyogram is measured by using a different device from this device, a conversion factor may be determined based on the levels of amplitude measured with respect to the same user by using the respective devices, for example, and the present specification should be read according to such a factor.

The above Embodiments are illustrated in such a manner that a biological signal cutting section is provided, where the biological signal cutting section cuts out the potential waveform of a masticatory electromyogram which is sent from the biological signal measurement section 50 in real time, based on the timing of menu item highlighting as a starting point. However, it is not essential for the biological signal cutting section to be provided. For example, the biological signal measurement section 50 may output results of measurement at the timing of menu item highlighting. Alternatively, the biological signal measurement section 50 may be incessantly transmitting results of measurement, and the masticatory electromyogram detector 10 may make a determination as to whether or not to receive the results of measurement based on the timing of menu item highlighting.

Moreover, before highlighting of a menu item, the highlight-type menu presentation section may present an indication on the output section 7 for helping the user to acknowledge that device manipulations are to be made based on chewing motions. For example, the following indication may be presented, for example: “Please make a chewing motion when a menu item you wish to select becomes highlighted”. This will help the user to acknowledge that he or she is using an interface system which allows a desired menu item to be selected from among a plurality of menu items when the user masticates.

Regarding the Embodiments above, the processes which have been described with reference to flowcharts can be implemented as a program which is executed by a computer. Such a computer program is distributed on the market in the form of a product recorded on a storage medium such as a CD-ROM, or transmitted through telecommunication lines such as the Internet. All or some of the constituent elements composing the masticatory electromyogram detector and the highlight-type menu presentation section may be implemented as a general-purpose processor (semiconductor circuit) executing a computer program. Alternatively, they may be implemented as a special processor in which such a computer program and a processor are integrated. A computer program which realizes the functions of the masticatory electromyogram detector may be executed by a processor which executes a computer program for realizing the functions of an interface system, or executed by another processor within the interface system. By being executed by a processor, such a computer program is able to control the outputting to the output section and the masticatory electromyogram measurement in the biological signal measurement section.

Although a channel switching manipulation for television broadcast programs is illustrated as an example in the description of each Embodiment above, this is only an example. The present invention is applicable not only to manipulations of television screens, but also to manipulations of other devices. For example, the present invention is applicable to a case where items to be cooked are displayed in order or randomly on the display of a microwave oven or other home appliance devices.

With a masticatory electromyogram detector according to the present invention and an interface system in which the masticatory electromyogram detector is incorporated, a user is able to rapidly select a highlighted menu item based on a potential which is measured at sites that naturally come into contact when a pair of glasses are worn. Manipulations of a wearable device (a head-mount display or a music player) whose interface sections such as buttons are small and manipulation inputs are difficult can be realized in a handsfree manner.

While the present invention has been described with respect to preferred embodiments thereof, it will be apparent to those skilled in the art that the disclosed invention may be modified in numerous ways and may assume many embodiments other than those specifically described above. Accordingly, it is intended by the appended claims to cover all modifications of the invention that fall within the true spirit and scope of the invention.

What is claimed is:

1. An interface system comprising:
an output section for visually presenting a manipulation menu for a device;
a measurement section for measuring a masticatory electromyogram of a user;
a menu presentation section for causing menu items of the manipulation menu to be presented in order via the output section;
an amplitude calculation section for determining a maximum amplitude of a potential waveform of the masticatory electromyogram;
a latency calculation section for determining a latency based on a point of highlighting each menu item as a starting point, the latency being an amount of time in which the potential waveform arrives at the maximum amplitude; and
a determination section for determining whether the maximum amplitude is greater than a previously determined threshold value and the latency is approximately 200 ms or not, wherein,
in accordance with a result of determination by the determination section, the menu presentation section executes a process corresponding the highlighted menu item.

2. The interface system of claim 1, wherein,
if the determination section determines that the maximum amplitude is greater than the previously determined threshold value and the latency is in the neighborhood of 200 ms,
it is determined that the masticatory electromyogram has been intentionally issued, and the menu presentation section executes the process corresponding to the highlighted menu item.

3. The interface system of claim 1, wherein, the measurement section measures the masticatory electromyogram based on a potential difference measured with electrodes worn on a nasion and a mastoid of the user.

4. The interface system of claim 1, further comprising a cut-out section for cutting out a potential waveform of the masticatory electromyogram in accordance with highlighting of each menu item by the menu presentation section, wherein
the amplitude calculation section determines the maximum amplitude of the potential waveform having been cut out.

5. The interface system of claim 4, wherein, the cut-out section at least cuts out the potential waveform a time slot including 150 ms to 250 ms after highlighting.

6. The interface system of claim 2, wherein, if the maximum amplitude is greater than the previously determined threshold value and the latency is in the neighborhood of 200 ms, the determination section determines that the measured masticatory electromyogram has been intentionally issued with respect to the highlighting of the menu item, and if the maximum amplitude is greater than the previously determined threshold value but the latency is not in the neighborhood of 200 ms, the determination section determines that the measured masticatory electromyogram has not been intentionally issued with respect to the highlighting of the menu item.

7. The interface system of claim 2, wherein, based on whether the maximum amplitude is greater than 50 μV as the previously determined threshold value and the latency is in the neighborhood of 200 ms or not, the determination section determines that the measured masticatory electromyogram has been intentionally issued with respect to the highlighting of the menu item.

8. An interface system comprising:
an output section for visually presenting a manipulation menu for a device;
a measurement section for measuring a masticatory electromyogram of a user;
a menu presentation section for causing menu items of the manipulation menu to be presented in order via the output section;
an amplitude calculation section for determining a maximum amplitude of a potential waveform of the masticatory electromyogram;
a latency calculation section for determining a latency based on a point of highlighting each menu item as a starting point, the latency being an amount of time in which the potential waveform arrives at the maximum amplitude;
a determination section for acquiring an average value and a variance value of each of maximum amplitude and latency of the user, and determining that the measured masticatory electromyogram has been intentionally issued with respect to the highlighting of the menu item if the maximum amplitude of the potential waveform of the measured masticatory electromyogram is contained in a range defined by the acquired average value of maximum amplitude±a standard deviation and the latency of the measured masticatory electromyogram is contained in a range defined by the acquired average value of latency±a standard deviation, wherein,
in accordance with a result of determination by the determination section, the menu presentation section executes a process corresponding the highlighted menu item.

9. An interface system comprising:
an output section for visually presenting a manipulation menu for a device;
a measurement section for measuring a masticatory electromyogram of a user;
a menu presentation section for causing menu items of the manipulation menu to be presented in order via the output section;
an amplitude calculation section for determining a maximum amplitude of a potential waveform of the masticatory electromyogram;
a latency calculation section for determining a latency based on a point of highlighting each menu item as a starting point, the latency being an amount of time in which the potential waveform arrives at the maximum amplitude;
a determination section for acquiring maximum amplitude and latency of the user, and determining that the measured masticatory electromyogram has been intentionally issued with respect to the highlighting of the menu item, by using ranges which are defined by the acquired maximum amplitude ±50 μV and the acquired latency ±50 ms as detection target ranges for determining whether the masticatory electromyogram has been intentionally issued by the user or not.

10. The interface system of claim 2, wherein the menu presentation section switches an order of menu item highlighting between descending and random,
the interface system further comprising a switching section for acquiring information as to which one of the descending order and the random order has been adopted in the menu presentation section, and switching detection target ranges for the determination section to determine whether the masticatory electromyogram has been intentionally issued by the user or not.

11. The interface system of claim 10, wherein, if the order of menu item highlighting is random, the switching section switches the detection target ranges for maximum amplitude and latency to be 200±70 μV and 280±50 ms, respectively.

12. The interface system of claim 1, further comprising a range determination section for retaining an elapsed time since a menu item was last presented by the menu presentation section, and for updating detection target ranges in a direction of expanding the detection target ranges if a certain amount of time or more has elapsed, the detection target ranges being used by the determination section to determine whether the masticatory electromyogram has been intentionally issued by the user or not.

13. The interface system of claim 12, wherein, if the masticatory electromyogram is detected after the detection target ranges are expanded by the range determination section, the range determination section newly sets a maximum amplitude and a latency value of the detected masticatory electromyogram as central values of the detection target ranges.

14. A method to be executed in an interface system, the method comprising the steps of:
visually presenting a manipulation menu for a device;
receiving a measured masticatory electromyogram of a user;
presenting menu items of the manipulation menu in order via the output section;
determining a maximum amplitude of a potential waveform of the masticatory electromyogram;
determining a latency based on a point of highlighting each menu item as a starting point, the latency being an amount of time in which the potential waveform arrives at the maximum amplitude;
determining whether the maximum amplitude is greater than a previously determined threshold value and the latency is approximately 200 ms or not; and
executing a process corresponding to the highlighted menu item in accordance with a result of determination by the determining step.

15. A computer program embodied in a computer-readable medium and to be executed by a computer,
the computer program causing the computer to execute the steps of:
visually presenting a manipulation menu for a device;
receiving a measured masticatory electromyogram of a user;
presenting menu items of the manipulation menu in order via the output section;
determining a maximum amplitude of a potential waveform of the masticatory electromyogram;
determining a latency based on a point of highlighting each menu item as a starting point, the latency being an amount of time in which the potential waveform arrives at the maximum amplitude;
determining whether the maximum amplitude is greater than a previously determined threshold value and the latency is approximately 200 ms or not; and
executing a process corresponding to the highlighted menu item in accordance with a result of determination by the determining step.
